(19) United States
(12) Patent Application Publication
(10) Pub. No.: US 2008/0051449 A1
(43) Pub. Date: Feb. 28, 2008

(54) PREPARATION OF ATORVASTATIN CALCIUM FORM VI AND COMPOSITIONS THEREOF

(76) Inventors: Sudeep Kumar Agrawal, Hyderabad (IN); Pratibha Kuldeep Bhat, Pune (IN); Srinivasulu Gudipati, Hyderabad (IN); Srinivas Katkam, Secunderabad (IN); Satyanarayana Komati, Hyderabad (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/668,608

(22) Filed: Jan. 30, 2007

Related U.S. Application Data
(60) Provisional application No. 60/803,136, filed on May 25, 2006.

(30) Foreign Application Priority Data
Jan. 30, 2006 (IN) 144/CHE/2006

(51) Int. Cl. A61K 31/40; A61P 9/00; C07D 207/30

(52) U.S. Cl. 514/423; 548/537

(57) ABSTRACT

The present invention provides processes to prepare atorvastatin calcium Form VI, pharmaceutical compositions comprising atorvastatin calcium Form VI, and processes to prepare said compositions. An aspect of the invention relates to atorvastatin calcium Form VI compositions having enhanced bioavailability when administered orally to humans.

### INTRODUCTION TO THE INVENTION

The present invention relates to processes to prepare atorvastatin calcium Form VI, pharmaceutical compositions comprising atorvastatin calcium Form VI and processes to prepare said compositions.

Further, the present invention relates to processes for the preparation of atorvastatin calcium crystalline Form VI from its other polymorphic forms, pharmaceutical compositions comprising atorvastatin calcium Form VI, and processes to prepare said compositions.

Atorvastatin calcium is chemically known as [R—(R*,R*)]-2-(4-fluorophenyl)-β,δ-dihydroxy-5-(1-methylethyl)-3-phenyl-4[(phenyl amino) carbonyl]-IH-pyrrole-1-heptanoic acid, calcium salt (2:1) trihydrate (which is hereinafter referred to by its adopted name “atorvastatin calcium”), and is represented by Formula I.

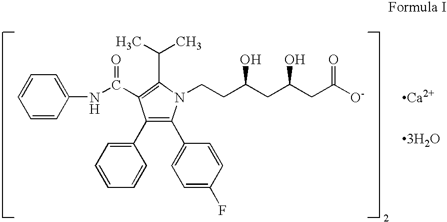

Atorvastatin calcium is used as a lipid-lowering agent for the treatment of hypercholesterolemia and is marketed as the hemicalcium salt trihydrate under the brand name LIPITOR® in the form of tablets containing 10, 20, 40 or 80 mg equivalent of atorvastatin.

U.S. Pat. No. 5,929,156 discloses crystalline Form I of atorvastatin calcium hydrate, oral formulations comprising it, crystalline Form II atorvastatin calcium and hydrates thereof and crystalline Form IV atorvastatin calcium and hydrates thereof. LIPITOR® tablets comprise crystalline Form I of atorvastatin calcium.

U.S. Pat. No. 6,121,461 discloses crystalline Form III of atorvastatin calcium hydrate, which is also useful as hypolipidemic and hypocholesterolemic agent.

International Application Publication No. WO 01/36384 discloses the Form V of atorvastatin calcium and hydrates thereof, its preparation and its pharmaceutical compositions.

International Application Publication No. WO 03/011826, U.S. Patent Application Publication No. 2004/0242899 and U.S. Pat. No. 7,074,818 disclose crystalline forms VI and VII of atorvastatin calcium and processes for preparing these forms.

The calcium salt of atorvastatin enables atorvastatin to be conveniently formulated in, for example, tablets, capsules, lozenges, powders, and the like for oral administration. Additionally, there is a need to produce atorvastatin in a pure and crystalline form to enable formulations meeting stability, dissolution and bioavailability requirements.

U.S. Food and Drug Administration (“FDA”) draft guidance, “Guidance for Industry ANDAs: Pharmaceutical Solid Polymorphism Chemistry, Manufacturing, and Controls Information” states clearly that polymorphic forms of a drug substance can have different chemical and physical properties, including melting point, chemical reactivity, apparent solubility, dissolution rate, optical and mechanical properties, vapor pressure, and density. These properties can have a direct effect on the ability to process and/or manufacture the drug substance and the drug product, as well as on drug product stability, dissolution, and bioavailability. Thus, polymorphism can affect the quality, safety, and efficacy of the drug product. Special focus is suggested when such various polymorphic forms show differences in physicochemical and mechanical properties.

Over the years, the FDA has approved a number of Abbreviated New Drug Applications in which the drug substance in the generic drug product had a different polymorphic form from the drug substance in the respective reference listed drug (RLD).

There is no predictive tool to determine and establish the desired physicochemical and mechanical properties of a different polymorphic form of active substance that is used to formulate a generic drug product that will have pharmaceutical and bio equivalence with the reference listed drug.

Hence there is a great felt need to identify and establish the physicochemical properties of atorvastatin calcium Form VI and to formulate with suitable compositions to show pharmaceutical and bio equivalence with that of reference listed drug.

Furthermore, the process to prepare atorvastatin calcium polymorph needs to be the one that is amenable to large-scale production. Additionally, it is desirable that the product should be in a form that is readily filterable and easily dried. It is also economically desirable that the product be stable for extended periods of time without the need for specialized storage conditions.

The present invention addresses the above-mentioned and other such considerations.

### SUMMARY OF THE INVENTION

An aspect of the present invention provides processes to prepare atorvastatin calcium Form VI with desired physicochemical properties.

Another aspect of the present invention provides pharmaceutical compositions comprising atorvastatin calcium Form VI, and processes to prepare said compositions.

A further aspect of the present invention provides pharmaceutical compositions comprising atorvastatin calcium Form VI, wherein said compositions exhibit improved bioavailability after oral administration to humans, as compared to commercially available LIPITOR® tablets.

In an embodiment, the present invention provides for process to prepare crystalline atorvastatin calcium Form VI from its other polymorphic forms, including Form I and Form VII.

In another embodiment, the stable atorvastatin calcium Form VI of the present invention has a particle size distribution (D₉₀) less than about 50 μm.

In still further embodiment, the pharmaceutical compositions of present invention comprise stable atorvastatin calcium Form VI with desired physicochemical properties, such compositions exhibiting a required in vitro dissolution profile.

An aspect of the invention provides a process for preparing crystalline Form VI of atorvastatin calcium, comprising heating Form VII of atorvastatin calcium at about 65-70° C. until a moisture content about 2 weight percent, or less, is obtained.

Another aspect of the invention provides a process for preparing crystalline Form VI of atorvastatin calcium, comprising crystallizing atorvastatin calcium from a solution comprising atorvastatin calcium, acetonitrile, and water.

In a further aspect, the invention provides a solid pharmaceutical composition comprising atorvastatin calcium Form VI and about 4 to about 10 percent by weight of a nonionic surfactant.

A still further aspect of the invention provides a crystalline Form VI of atorvastatin calcium having a particle size distribution D₉₀ less than about 50 μm.

### BRIEF DESCRIPTION OF THE DRAWINGS

FIG. 1 is an X-ray powder diffraction pattern of atorvastatin calcium crystalline Form VI prepared in Example 1.

FIG. 2 is an X-ray powder diffraction pattern of atorvastatin calcium crystalline Form VI prepared in Example 2.

FIG. 3 is an infrared absorption spectrum of atorvastatin calcium crystalline Form VI prepared in Example 3.

FIG. 4 is a differential scanning calorimetric curve of atorvastatin calcium crystalline Form VI prepared in Example 3.

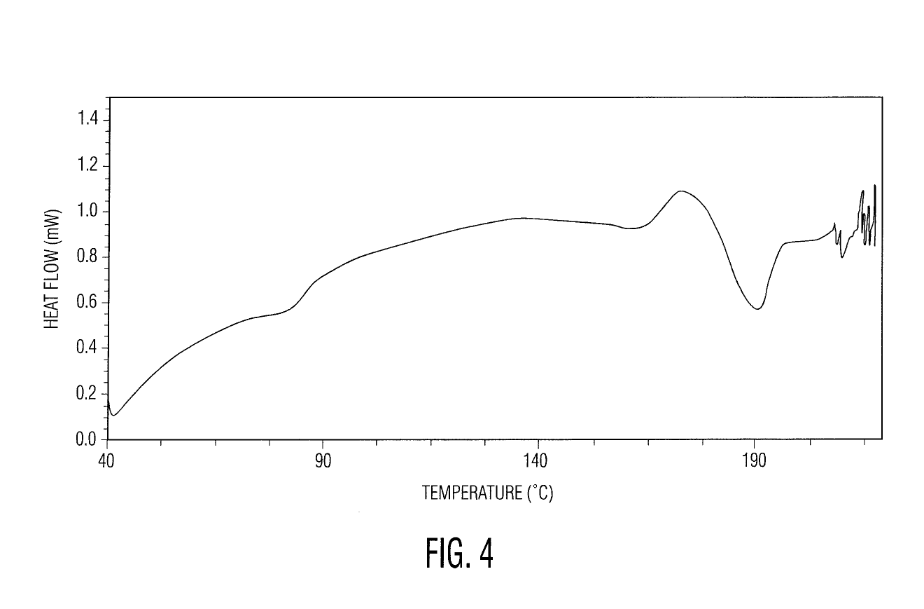

FIG. 5 shows comparative X-ray powder diffraction patterns of: Example 8 tablets, (“A”); atorvastatin calcium crystalline Form VI of Example 3, (“B”); and “placebo” tablets of Example 8 (omitting the drug compound), (“C”).

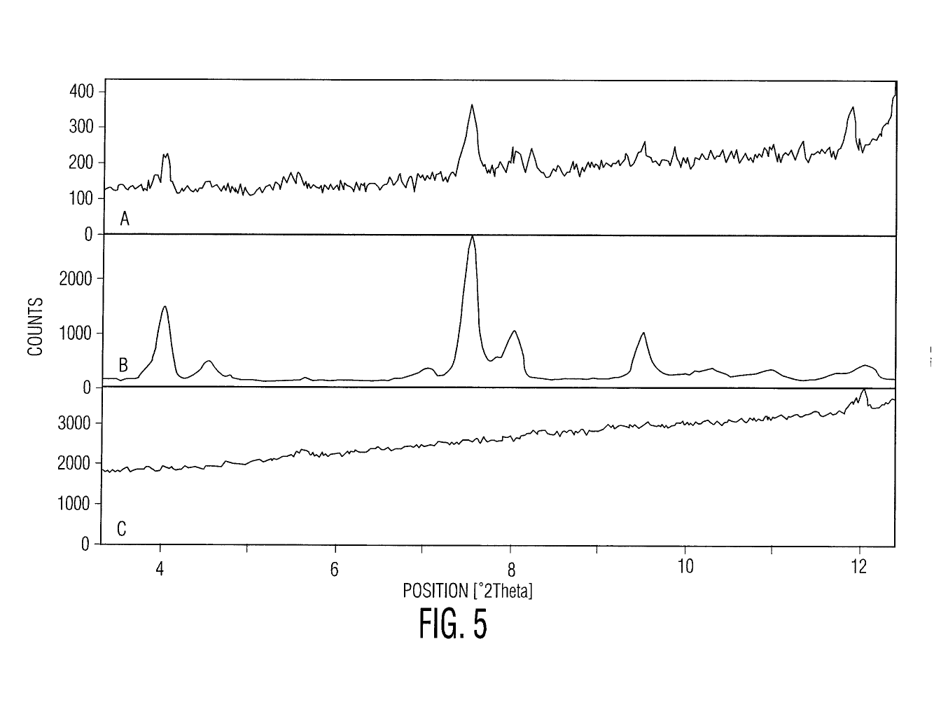

### DETAILED DESCRIPTION OF THE INVENTION

The present invention relates to processes for the preparation of atorvastatin calcium crystalline Form VI from its other polymorphic forms, pharmaceutical compositions comprising atorvastatin calcium Form VI and processes to prepare said compositions.

In an embodiment, the present invention provides processes to prepare atorvastatin calcium Form VI with desired physicochemical properties.

In a further embodiment, the present invention provides processes to prepare crystalline atorvastatin calcium Form VI from its other polymorphic forms.

In another aspect of the present invention, there is provided a process for the preparation of atorvastatin calcium Form VI from other crystalline forms of atorvastatin calcium, the process comprising crystallizing atorvastatin calcium from a solution comprising acetonitrile and water.

The solution of atorvastatin calcium can be prepared by dissolving atorvastatin calcium in acetonitrile and water or obtaining such a solution form a previous processing step wherein atorvastatin calcium is formed.

When the solution is prepared by dissolving atorvastatin calcium in acetonitrile and water, any form of atorvastatin calcium such as any crystalline or amorphous form, including any salts, solvates and hydrates may be utilized for preparing the solution. Mixtures of different forms are also useful in the process.

The concentration of the solute can range from about 40 g/l to about 100 g/l in the solution. Any other concentration may be used as long as a clear solution is obtained, and the maximum solubility will, of course, differ according to the ratio of acetonitrile to water and the temperature that is being used. Generally, the higher concentrations will promote greater product recoveries.

The amount of water that is used can range from about 1 to about 5 times, or about 1.5 to about 2.5 times, a volume of the acetonitrile used.

Suitable temperatures for providing the solution of atorvastatin calcium range form about 20 to 120° C., or about 80 to 100° C. Any temperature can be used to obtain a clear solution as long as it does not affect the quality of atorvastatin calcium.

The clear solution obtained can be optionally treated with activated charcoal to enhance the color of the compound, followed by filtration through an inert medium such as a flux calcined diatomaceous earth (Hyflow) bed to remove the carbon, or by using other clarification techniques known to those skilled in the art.

Depending upon the equipment used and the concentration and temperature of the solution, the filtration apparatus may need to be preheated to avoid premature crystallization.

To enhance the crystallization, the mass may be further maintained at temperatures lower than the temperatures at which the solution is prepared, such as for example about 1° C. to about 40° C., or about 20° C. to about 30° C., for a period of time as required for a more complete isolation of the product. The exact cooling temperature and time required for complete crystallization can be readily determined by a person skilled in the art and will also depend on parameters such as concentration and temperature of the solution or slurry.

Crystalline Form VI of atorvastatin calcium can be recovered from the slurry by using any of the techniques such as filtration by gravity or by suction, decantation, centrifugation, and the like.

The wet solid thus obtained can be dried using any technique such as fluid bed drying (FBD), spin flash drying, aerial drying, and oven drying under ambient or reduced pressure. For example, drying can be performed under reduced pressure or under atmospheric pressure at a temperature of at about 40° C. to 120° C., or 80° C. to 90° C. The atmosphere for drying can be air or a partially or completely inert atmosphere, such as by using nitrogen.

In yet another aspect, the present invention provides a process for the preparation of atorvastatin calcium crystalline Form VI from atorvastatin calcium crystalline Form VII comprising heating Form VII of atorvastatin calcium at a suitable temperature to produce a moisture content about 2% w/w, or less.

The atorvastatin calcium crystalline Form VII can be heated using any technique such as fluid bed drying (FBD), spin flash drying, aerial drying, and oven drying under ambient or reduced pressure. For example, drying can be performed under reduced pressure or under atmospheric pressure at a temperature of about 40° C. to 120° C., or about 60° C. to 80° C. The atmosphere for drying can be air or a partially or completely inert atmosphere, such as by using nitrogen.

Suitably the heating process can be carried out from about 10 hours to about 30 hours, or longer, depending the drying technique used and the choice of the temperature. The conversion of atorvastatin calcium crystalline Form VII to atorvastatin calcium crystalline Form VI can be confirmed by analyzing the moisture content of atorvastatin calcium crystalline Form VI. The heating process can be terminated as soon as the moisture content becomes about 2% by weight, or less.

The atorvastatin calcium crystalline Form VI obtained by the above methods is characterized by its X-ray powder diffraction (“XRPD”) pattern, using copper Kα-1 radiation, with peaks at about 3.9, 4.5, 7.5, 7.8, 9.2, and 18.8, ±0.2 degrees 2-theta. All XRPD patterns reported herein were generated using copper Kα-1 radiation.

The crystalline Form VI of atorvastatin calcium of the present invention exhibits physical properties like differential scanning calorimetry, infrared spectrum, bulk density, particle size distribution and flowability which are distinct when compared to other crystalline forms.

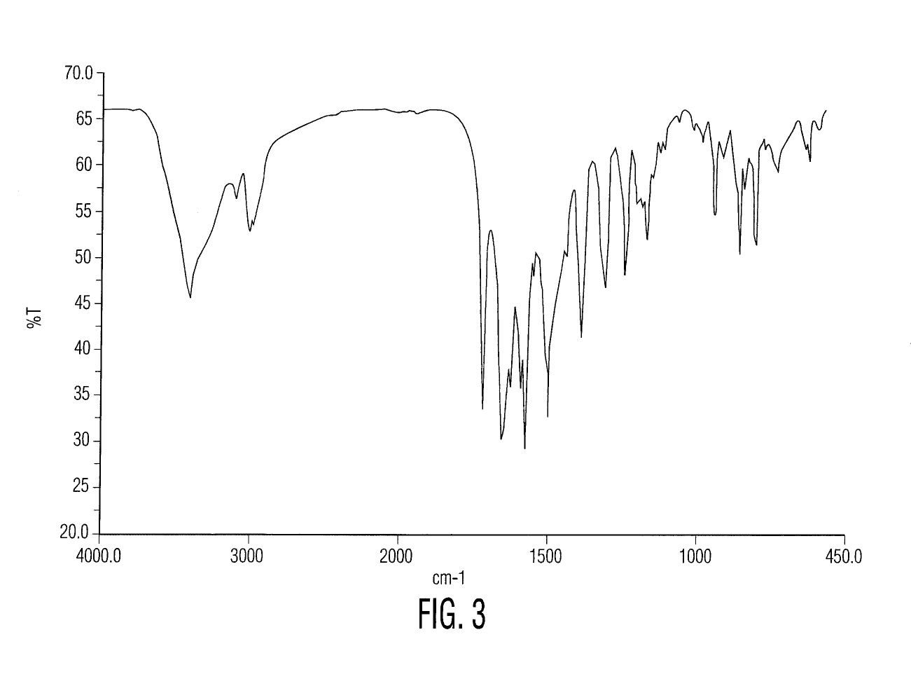

Crystalline Form VI of atorvastatin calcium produced by the present invention has a differential scanning calorimetry curve having an endotherm at about 164 to about 190° C. and a water content by the Karl Fischer (“KF”) method about 2% w/w, or between about 1 and about 2 percent by weight. It is further characterized by an infrared absorption spectrum substantially in accordance with FIG. 3.

The process of the present invention is simple, inexpensive, eco-friendly, commercially suitable and reproducible on an industrial scale.

A particle size distribution of D₅₀ as used herein is defined as the distribution where 50 volume percent of the particles are smaller than that size given. A particle size distribution of D₁₀ as used herein is defined as the distribution where 10 volume percent of the particles are smaller than that size given. A particle size distribution of D₉₀ as used herein is defined as the distribution where 90 volume percent of the particles are smaller than that size given. The D₅₀ value is considered to be a “mean particle size.” Particle size can be determined using commercially available instrumentation, such as that sold by Malvern Instruments Ltd. of Malvern, Worcestershire, United Kingdom and using a laser diffraction principle.

The particle size distribution D₉₀ of atorvastatin calcium Form VI produced by the present invention is in the range of about 0.1 μm to about 50 μm. In the context of present invention, the stable atorvastatin calcium Form VI has a particle size distribution D₉₀ less than about 50 μm.

Untapped bulk density of a substance is the undisturbed packing density of that substance and tapped bulk density relates to the packing density after tapping a bed of substance until no change in the packing density is seen. Bulk density and tapped density can be determined using compendial bulk density apparatus, the method being given in United States Pharmacopeia 29, United States Pharmacopeial Convention, Inc., Rockville, Md., 2005, at pages 2638-2639.

The Hausner ratio is a measure of inter-particle friction and the potential powder arch or bridge strength and stability (H. H. Hausner, “Friction conditions in a mass of metal powders,” International Journal of Powder Metallurgy 1967, Vol. 3(4), pages 7-13). It has been widely used to estimate the flow properties of powders, blends, granules and other such particles or aggregates and is expressed as the ratio of tapped bulk density to the untapped bulk density of the substance.

Atorvastatin calcium crystalline Form VI produced by the present invention has a bulk density that varies from about 0.05 to about 0.15 g/ml, while its tapped density ranges between about 0.15 and about 0.4 g/ml. The Hausner ratio varies in the range of about 0.5 to about 3.5, or about 1 to about 2.

In another embodiment, the present invention provides for pharmaceutical compositions comprising atorvastatin calcium Form VI, and processes to prepare said compositions.

Atorvastatin calcium Form VI may be formulated into pharmaceutical compositions for oral, parenteral, topical or other known routes of drug delivery using methods known to one skilled in the art. The pharmaceutical compositions comprising atorvastatin calcium Form VI may exhibit immediate or modified release characteristics.

The amount of atorvastatin calcium Form VI in the dosage form may vary from about 5 mg to about 120 mg, or about 10 to about 80 mg, of atorvastatin equivalent.

The compositions of the present invention may comprise pharmaceutically acceptable excipients that are non-toxic to the mammal intended to be treated when the composition is administered in an amount effective to treat the mammal, such as, but not limited to, diluents, binders, disintegrants, surfactants, tableting aids, colorants, anti-oxidants, sweeteners and film-forming agents.

Common diluents useful in the compositions of the present invention include, but are not limited to, microcrystalline cellulose, silicified microcrystalline cellulose, micro-fine cellulose, lactose, lactose monohydrate, pre-gelatinized starch, calcium carbonate, calcium sulfate, sugar, dextrates, dextrin, dextrose, sorbitol, dibasic calcium phosphate dihydrate, tribasic calcium phosphate, kaolin, magnesium carbonate, magnesium oxide, maltodextrin, polymethacrylates, and mixtures thereof.

Binders useful in the compositions of the present invention include, but are not limited to, starch, microcrystalline cellulose, cellulose ethers like methyl cellulose, ethyl cellulose; hydroxypropyl cellulose (also available under the brand name KLUCEL™ EF, LF and EXF); sodium carboxymethylcellulose, dextrose, sucrose, sorbitol, polyethylene glycol, polyvinylpyrrolidone, pectin, polyacrylamides, polyvinyloxoazolidone, polyvinylalcohol and mixtures thereof.

Disintegrants useful in the compositions of the present invention include, but are not limited to, croscarmelose sodium, crospovidone, cellulose ethers, starch and sodium starch glycolate.

Lubricants and glidants useful in the compositions of the present invention include, but are not limited to, colloidal silicon dioxide, stearic acid, magnesium stearate, calcium stearate, talc, hydrogenated castor oil, sucrose esters of fatty acid, microcrystalline wax, yellow beeswax and white beeswax.

Surfactants that may be useful in the composition of the present invention include but are not limited to anionic surfactants like potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (for example, cholic acid, deoxycholic acid, glycocholic acid, taurocholic acid and glycodeoxycholic acid) and salts thereof (for example, sodium deoxycholate); cationic surfactants like quaternary ammonium compounds (for example, benzalkonium chloride, cetyltrimethylammonium bromide, lauryldimethylbenzylammonium chloride, acyl carnitine hydrochlorides and alkyl pyridinium halides); nonionic surfactants like polyoxyethylene fatty alcohol ethers (also called Macrogol™ and Brij™), polyoxyethylene sorbitan fatty acid esters (polysorbates or Tweens™), polyoxyethylene fatty acid esters (Myrj™), sorbitan esters (Span™), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, methylcellulose, hydroxycellulose, hydroxy propylcellulose, hydroxy propylmethylcellulose, noncrystalline cellulose, polyvinyl alcohol, and polyvinylpyrrolidone.

Antioxidants that are useful in the composition of the present invention include but are not limited to butylated hydroxanisole, sodium ascorbate, butylated hydroxytoluene, sodium metabisulfate, malic acid, citric acid and ascorbic acid.

The compositions of the present invention may further comprise metal salts including but not limited to sodium bicarbonate, sodium carbonate, calcium carbonate, magnesium carbonate, sodium hydroxide, calcium hydroxide, magnesium hydroxide, sodium silicate, calcium silicate, magnesium silicate, magnesium aluminate, aluminium magnesium hydroxide, sodium phosphate, disodium orthophosphate and sodium dihydrogen phosphate and oxides of alkaline earth metal salts.

Useful organic alkalizers or inorganic salts that may be used in the compositions for pH adjustment include but are not limited to sodium or potassium carbonate; sodium or potassium citrate; sodium or potassium acetate, and basic amines such as arginine, tromethamine and meglumine. and oxides of alkaline metal earth salts.

In an aspect of the invention, the atorvastatin calcium Form VI is formulated into tablets. Such tablets may be prepared by wet granulation, dry granulation, direct compression or any other processes known in the art. Optionally, the tablets may be coated.

In an embodiment of the present invention, pharmaceutical compositions comprising atorvastatin calcium Form VI exhibit improved bioavailability as compared to commercially available LIPITOR® tablets after oral administration to human subjects under fasted conditions. This improvement in bioavailability can reduce the administrable dose of atorvastatin without compromising its therapeutic efficacy. Moreover, with a reduced dose, the adverse effects of atorvastatin as well as the medication cost can also be minimized.

Improved bioavailability can be achieved by incorporating about 4 to about 10, or about 5 to about 10, percent by weight of an anionic surfactant into a composition comprising atorvastatin calcium. Generally, the nonionic surfactant will be in admixture with atorvastatin calcium and other solid components of a tablet, granule, etc.; in an embodiment of the invention a nonionic surfactant is incorporated into a granulating solution that is mixed with a drug-containing powder blend to form granulated particles, and the granulated particles can be compressed into tablets or filled into capsules to make a finished dosage form.

Some chemical types of nonionic surfactants that have found use in food and drug products include, without limitation thereto, polyoxyethylene alkylphenols, polyoxyethylene alcohols, and polyoxyethylene esters of fatty acids. The more commonly used anionic surfactants include polysorbates, or polyoxyethylene sorbitan fatty acid esters, which are reaction products of polyethoxylated sorbitan and a longer-chain (e.g., C₁₂₋₂₂) fatty acid. Examples of specific polysorbates that are frequently used in pharmaceutical formulations include, but are not limited to, polysorbate 20 (polyoxyethylene (20) sorbitan monolaurate, sold as TWEEN™ 20), polysorbate 40 (polyoxyethylene (20) sorbitan monopalmitate, sold as TWEEN™ 40), polysorbate 60 (polyoxyethylene (20) sorbitan monostearate, sold as TWEEN™ 60), polysorbate 80 (polyoxyethylene (20) sorbitan monooleate, sold as TWEEN™ 80), and polysorbate 120 (polyoxyethylene (20) sorbitan isostearate). In each of these products, the sorbitan is reacted to contain 20 polyoxyethylene groups, prior to ester formation. TWEEN™ is a trademark of Uniquema, a unit of Croda, Yorkshire, United Kingdom. Similar products are sold by other companies, using different trademarks, and other mono- and tri-esters containing 20 or fewer molar equivalents of the polyoxyethylene groups are also available.

The pharmaceutical compositions comprising atorvastatin calcium Form VI of the present invention can be used for the treatment of hypercholesterolemia.

The following examples are provided only for the purpose of illustrating certain specific aspects and embodiments of the present invention and should not be construed as limitations on the scope or spirit of the invention.

### EXAMPLE 1

### Preparation of Atorvastatin Calcium Crystalline Form VI from Atorvastatin Calcium Crystalline Form I

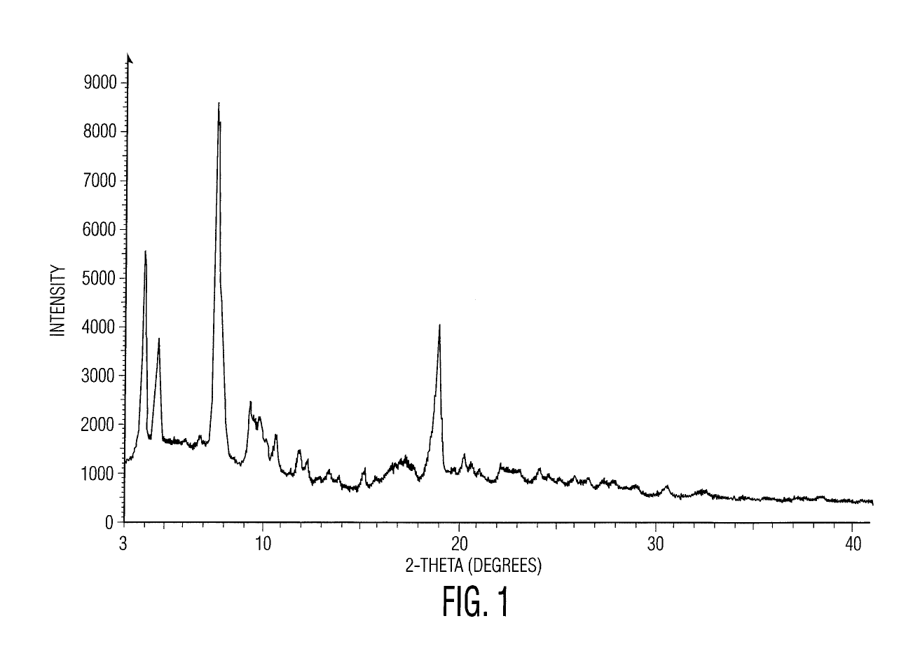

20 g of crystalline Form I of atorvastatin calcium was charged into a 1-liter round bottom flask containing a mixture of acetonitrile (100 ml) and water (200 ml). The contents were heated to reflux and stirred at a temperature of about 78 to 79° C. with continued stirring for a period of about 22 hours. Heating was stopped and the solution was cooled to 25-35° C. with stirring for about 1 hour. The separated solid was filtered and washed with 40 ml of water. The wet solid material was transferred into an oven and dried at a temperature of about 65° C. under a vacuum of about 600-700 mm Hg for a period of about 24 hours to yield 17.5 g of the title compound having an XRPD pattern substantially in accordance with FIG. 1 and water by KF of 1.2% w/w.

### EXAMPLE 2

### Preparation of Atorvastatin Calcium Crystalline Form VI from Atorvastatin Calcium Crystalline Form VII

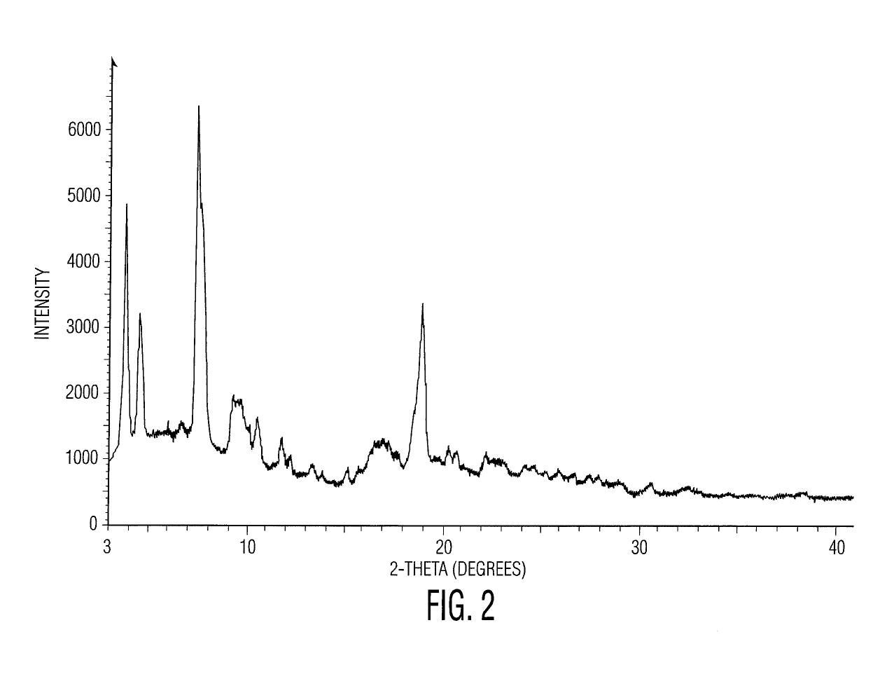

5.0 g of atorvastatin calcium crystalline Form VII was kept in a clean and dry oven followed by subjecting to drying at about 65 to 70° C. under a vacuum of about 600-700 mm Hg over a period of about 16 hours to afford 4.9 g of the desired title compound having an XRPD pattern substantially in accordance with FIG. 2 and water by KF of 1.58% w/w.

### EXAMPLE 3

### Preparation of Atorvastatin Calcium Crystalline Form VI

360 liters of acetonitrile and 24 kg of atorvastatin was taken into a reactor and subjected to heating to 44° C. for 1 hour. 73 liters of 10% aqueous calcium acetate solution was added to the reaction mass and maintained for 1 hour, 15 minutes. 4.5 liters of 10% aqueous sodium hydroxide was added to the above reaction mass and subjected to heating to reflux at 70° C. for 1 hour. The reaction mass was filtered and the solid washed with 45 liters of acetonitrile. 9 liters of 10% sodium hydroxide was added to the above solid and subjected to heating to reflux at 70° C. for a period of about 8 hours. The reaction mass was cooled to 30° C. for a period of about 1.5 hours and centrifuged followed by washing with 112.5 liters of water. The obtained solid material was spin dried for a period of about 2 hours and kept for aerial drying for 30 minutes. The obtained solid material was dried at 55° C., cooled and the solid was thoroughly mixed. Finally the obtained solid material was subjected to rotatory cone vacuum drying to afford 35.8 kg (84.35%) of crystalline Form VI of atorvastatin calcium.

The particle size distribution of atorvastatin calcium Form VI was determined using a Malvern® particle size analyzer, and 0.7% of dioctyl sodium sulfosuccinate in n-hexane was used as the dispersant.

| Parameter | Particle Size (μm) |
|---|---|
| D₁₀ | 1.32 |
| D₅₀ | 5.22 |
| D₉₀ | 24.54 |

### EXAMPLE 4

### Tablets Comprising Atorvastatin Calcium Form VI

| Ingredient | Quantity/Batch (Kg) |
|---|---|
| Atorvastatin calcium Form VI | 0.42 |
| (Example 3) | |
| Microcrystalline cellulose | 3.13 |
| Lactose monohydrate | 0.81 |
| Sodium starch glycolate | 0.22 |
| SYLOID ™ AL-1 FP* | 0.33 |
| Sodium bicarbonate | 0.26 |
| Magnesium stearate | 0.03 |
| KLUCEL ™ LF** | 0.05 |
| Polysorbate 80 | 0.46 |
| Water | 1.6 |

COATING

| | |
|---|---|
| OPADRY ™ white OY-58900*** | 0.18 |
| Water | 1.8 |

*SYLOID contains precipitated silica; supplied by W. R. Grace & Co., U.S.A.
**KLUCEL LF is hydroxypropyl cellulose polymer of average molecular weight 95,000 and viscosity of 5% w/v aqueous solution is 75-150 mPa.
***OPADRY ™ White is a formulated film coating material sold by Colorcon, West Point, Pennsylvania U.S.A., that contains hydroxypropyl methylcellulose 2910/hypromellose 6 cps, titanium dioxide and talc.

Manufacturing Process:
1. Atorvastatin calcium (423.20 g), microcrystalline cellulose (1.88 kg), lactose monohydrate (0.805 kg), sodium starch glycolate (0.24 kg) and Syloid (0.300 kg) were sifted through an ASTM #40 mesh sieve.
2. The sifted ingredients of step 1 were blended in a rapid mixer granulator at a fast speed of the impeller for 15 minutes.
3. Polysorbate 80 and Klucel LF were dissolved in water.
4. The dry blend of step 2 was granulated using solution of step 3.
5. The granules were dried in a fluid bed dryer at an inlet temperature of 60° C. until a loss on drying below 2% w/w was achieved.
6. Microcrystalline cellulose (1.25 kg), sodium bicarbonate (0.234 kg), Syloid (0.027 kg), and sodium starch glycolate (0.216 kg) were sifted through an ASTM #40 mesh sieve.
7. The sifted ingredients of step 6 were mixed with granules of step 5 in a double cone blender for 20 minutes.
8. Magnesium stearate was sifted through an ASTM #60 mesh sieve and was blended with mixture of step 7 in a double cone blender for 5 minutes.
9. The blend was compressed using 21×10 mm modified capsule shaped punches to produce tablets containing 80 mg of atorvastatin.
10. The compressed tablets were coated with a dispersion of Opadry white.

Flow properties of atorvastatin calcium Form VI (Example 3) and lubricated granules of Example 4, step 8:

| | Atorvastatin | Lubricated |
|---|---|---|
| | calcium Form VI | Granules of |
| Parameter | (Example 3) | Example 4 |
| Bulk density (g/ml) | 0.099 | 0.54 |
| Tapped density (g/ml) | 0.195 | 0.69 |
| Hausner ratio | 1.98 | 1.28 |

Comparative in vitro dissolution testing of Example 4 tablets and LIPITOR® 80 mg tablets was performed with the following parameters:

Media: 0.1N hydrochloric acid (pH 1.2, 900 ml); Phosphate buffer pH 7.4 (900 ml).

Apparatus: USP apparatus type II (Paddle) from Test 711 “Dissolution” in United States Pharmacopeia 29, United States Pharmacopeial Convention, Inc., Rockville,

Speed: 50 rpm.

| | Drug Released (%) | | | |
|---|---|---|---|---|
| Time | 0.1N Hydrochloric Acid | | Phosphate Buffer pH 7.4 | |
| (minutes) | Example 4 | LIPITOR ® | Example 4 | LIPITOR ® |
| 0 | 0 | 0 | 0 | 0 |
| 10 | — | — | 80.8 | 94.2 |
| 20 | 33 | 35 | — | — |
| 30 | — | — | 94.2 | 98.7 |
| 40 | 42 | 43 | — | — |
| 60 | 45 | 48 | — | — |

### EXAMPLE 5

### Stability Study of Atorvastatin Calcium Form VI (Example 3) at 40° C. and 75% Relative Humidity

Stability packaging: The sample was packed in a clear polyethylene bag and flushed with nitrogen, then tied and placed in a black polyethylene bag along with silica gel and tied. Finally the black bag was placed in a triple laminated bag and sealed with a liner sealer.

| Time | Moisture Content | Total Impurities | Assay by HPLC |
|---|---|---|---|
| (Months) | (% w/w) | by HPLC (% w/w) | (% w/w) |
| Initial | 1.4 | 0.18 | 99.1 |
| 1 | 1.4 | 0.14 | 99.2 |
| 3 | 1.6 | 0.15 | 99.2 |
| 6 | 1.6 | 0.24 | 99.2 |

### EXAMPLE 6

### Stability Study of Atorvastatin Calcium Crystalline Form VI (Example 3) During Storage at 25° C. and 60% Relative Humidity.

Stability packaging: same as Example 5.

| Time | Moisture Content | Total Impurities by | Assay by HPLC |
|---|---|---|---|
| (Months) | (% w/w) | HPLC (% w/w) | (% w/w) |
| Initial | 1.4 | 0.18 | 99.1 |
| 1 | 1.5 | 0.16 | 99.1 |
| 3 | 1.7 | 0.14 | 99.1 |
| 6 | 1.5 | 0.23 | 99.3 |

### EXAMPLE 7

### Stability Study of Atorvastatin Calcium Crystalline Form VI (Example 3) Subject to Various Stress Conditions in an Open Container

| | | Total |
|---|---|---|
| | Water by KF | Impurities |
| Stress Condition | (% w/w) | (% w/w) |
| Initial analysis | 1.60 | 0.26 |
| Accelerated Stability (40° C. and 75% | 1.85 | 0.29 |
| relative humidity) 24 hours | | |
| Thermal (90° C.) 24 hours | 1.04 | 0.7 |
| UV Photostability λ = 254 nm, 24 hours | 1.54 | 0.27 |
| Compression (2000 kg/cm²) for 1 hour | 1.68 | 0.32 |

### EXAMPLE 8

### Tablets Comprising Atorvastatin Calcium Form VI (80 mg Atorvastatin)

| | |
|---|---|
| Ingredient | Quantity/Batch (g) |

GRANULATION

| | |
|---|---|
| Atorvastatin calcium Form VI (Example 3) | 12.7 |
| EUDRAGIT ™ EPO* | 6.4 |
| Methanol*** | 150 |
| Microcrystalline cellulose (AVICEL ™ PH112**) | 249.8 |
| Lactose monohydrate | 245.5 |

BLENDING AND LUBRICATION

| | |
|---|---|
| Atorvastatin calcium Form VI | 29.6 |
| Sodium starch glycolate | 30 |
| Sodium bicarbonate | 12 |
| Hydroxypropyl cellulose (KLUCEL ™ LF) | 8 |
| Magnesium stearate | 6 |

COATING

| | |
|---|---|
| OPADRY White | 15 |
| Water*** | 125 |

*Poly(butylmethacrylate), (2-dimethylaminoethyl)methacrylate, methylmethacrylate) 1:2:1; manufactured by Rohm and Hass Company, Philadelphia, Pennsylvania U.S.A.
**Manufactured by FMC Biopolymer Inc.
***Component evaporates during processing.

Manufacturing Process:
1. Microcrystalline cellulose and lactose were sifted through an ASTM # 40 mesh sieve and mixed well.
2. Atorvastatin calcium (first quantity) and Eudragit were dissolved in methanol with stirring to get a solution.
3. Mixture of step 1 was granulated with solution of step 2 in a fluid bed processor using the following parameters:
   Inlet temperature: 50-60° C.
   Product temperature: 30° C.
   Atomization air pressure: 1.4-1.6 bar.
4. The granules were dried at an inlet temperature of about 60° C. until a loss on drying (LOD) below 2% w/w at 105° C. was achieved.
5. The dried granules of step 4 were blended with the blending and lubrication ingredients atorvastatin calcium, sodium starch glycolate, sodium bicarbonate and hydroxypropyl cellulose.
6. Blend of step 5 was lubricated by adding magnesium stearate.
7. Lubricated blend of step 6 was compressed into tablets using a modified capsule shaped 21×10 mm punch set on a rotary compression machine at an average tablet weight of 1230 mg with an atorvastatin content of 80 mg.
8. Tablets of step 7 were coated with Opadry White dispersion in water.

In vitro dissolution testing of Example 8 tablets and LIPITOR® 80 mg tablets was performed with the following parameters:

Media: 0.1N hydrochloric acid (pH 1.2, 900 ml).

Apparatus: USP apparatus type II (Paddle) from Test 711 “Dissolution” in United States Pharmacopeia 29, United States Pharmacopeial Convention, Inc., Rockville, Maryland (2005).

Speed: 50 rpm.

| | |
|---|---|
| Time | Drug Released (%) |

| (minutes) | Example 8 | LIPITOR ® 80 mg |
|---|---|---|
| 0 | 0 | 0 |
| 20 | 33 | 30 |
| 40 | 40 | 40 |
| 60 | 42 | 45 |

### EXAMPLES 9-10

Tablet compositions comprising atorvastatin calcium Form VI (80 mg atorvastatin) showing enhanced bioavailability.

Quantity/Batch (g)

| | | |
|---|---|---|
| Ingredient | Example 9 | Example 10 |

GRANULATION

| | | |
|---|---|---|
| Atorvastatin calcium Form VI | 423.2 | 42.32 |
| Microcrystalline cellulose | 1876.8 | 187.3 |
| Lactose monohydrate | 805 | 125 |
| Sodium starch glycolate | 240 | 24 |
| Silicon dioxide | 300 | 30 |
| Hydroxypropyl cellulose | 80 | 8 |
| Polysorbate 80 | 465 | 2.4 |
| Acetonitrile* | — | 200 |
| Water* | 1600 | 100 |

BLENDING AND LUBRICATION

| | | |
|---|---|---|
| Microcrystalline cellulose | 1250 | 125 |
| Sodium bicarbonate | 260 | 26 |
| Silicon dioxide | 30 | 3 |
| Sodium starch glycolate | 240 | 24 |
| Magnesium stearate | 30 | 3 |

COATING

| | | |
|---|---|---|
| OPADRY White | 150 | 15 |
| Water* | 1250 | 125 |

*Component evaporates during processing.

Manufacturing Process:
1. Atorvastatin calcium, microcrystalline cellulose, lactose monohydrate, sodium starch glycolate and silicon dioxide were mixed together by blending.
2. Polysorbate 80 was dissolved in water (and acetonitrile, if any), and hydroxypropyl cellulose was dispersed in this solution.
3. Blend of step 1 was granulated using dispersion of step 2 in a rapid mixer granulator (Example 9) and a fluid bed processor (Example 10).
4. The granules were dried at an inlet temperature of about 60° C. until a loss on drying (LOD) below 2% w/w at 105° C. was achieved.
5. The dried granules of step 4 were blended with the blending and lubrication ingredients microcrystalline cellulose, sodium starch glycolate, sodium bicarbonate and silicon dioxide.
6. Blend of step 5 was lubricated by adding magnesium stearate.
7. Lubricated blend of step 6 was compressed into tablets using a modified capsule shaped 21×10 mm punch set on a rotary compression machine at an average tablet weight of 1230 mg with atorvastatin content of 80 mg.
8. Tablets of step 7 were coated with an Opadry dispersion in water.

Bioavailability results from an in vivo evaluation in 20 healthy human subjects under fasting conditions, using a 2-way crossover study design:

Test/Reference* Ratio (%)

| Pharmacokinetic Parameter | Example 9 | Example 10 |
|---|---|---|
| Cₘₐₓ | 186.06 | 133.67 |
| AUC₀₋ₜ | 155.44 | 113.11 |
| AUC_{0-∞} | 154.77 | 112.46 |

*LIPITOR ® tablets (80 mg atorvastatin)

We claim:

1. A process for preparing crystalline Form VI of atorvastatin calcium, comprising heating Form VII of atorvastatin calcium at about 65-70° C. until a moisture content about 2 weight percent, or less, is obtained.

2. The process of claim 1, wherein heating is conducted under a vacuum.

3. The process of claim 1, wherein a moisture content between about 1 weight percent and about 2 weight percent is obtained.

4. The process of claim 1, wherein heating is conducted under a vacuum and a moisture content between about 1 weight percent and about 2 weight percent is obtained.

5. A process for preparing crystalline Form VI of atorvastatin calcium, comprising crystallizing atorvastatin calcium from a solution comprising atorvastatin calcium, acetonitrile, and water.

6. The process of claim 5, wherein a volume of water is about 1 to about 5 times a volume of acetonitrile.

7. The process of claim 5, wherein a volume of water is about 1.5 to about 2.5 times a volume of acetonitrile.

8. A solid pharmaceutical composition comprising atorvastatin calcium Form VI and about 4 to about 10 percent by weight of a nonionic surfactant.

9. The solid pharmaceutical composition of claim 8, comprising about 5 to about 10 weight percent of a nonionic surfactant.

10. The solid pharmaceutical composition of claim 8, wherein a nonionic surfactant comprises a polyoxyethylene sorbitan fatty acid ester.

11. The solid pharmaceutical composition of claim 8, comprising about 5 to about 10 weight percent of a polyoxyethylene sorbitan fatty acid ester.

12. The solid pharmaceutical composition of claim 8, wherein a nonionic surfactant comprises polysorbate 80.

13. The solid pharmaceutical composition of claim 8, comprising about 5 to about 10 weight percent of polysorbate 80.

14. Crystalline Form VI of atorvastatin calcium having a particle size distribution D₉₀ less than about 50 μm.
